(19) United States
(12) Patent Application Publication
(10) Pub. No.: US 2024/0382297 A1
(43) Pub. Date: Nov. 21, 2024

(54) DENTAL APPARATUS FOR AIR ABRASION AND POLISHING

(71) Applicant: ZYGMUNT BARAN, Windsor (CA)

(72) Inventor: ZYGMUNT BARAN, Windsor (CA)

(21) Appl. No.: 18/695,882

(22) PCT Filed: Oct. 28, 2021
(86) PCT No.: PCT/CA2021/000097

§ 371 Date: Mar. 27, 2024

(51) Int. Cl. A61C 17/022; A61C 17/02

(52) U.S. Cl. CPC A61C 17/022; A61C 17/0202; A61C 17/0208; A61C 17/0217

(57) ABSTRACT

A dental apparatus for generating a jet of gas mixed with solid powder particles, comprised of a hand piece connected to a powder feeder, and attached by a supply cable to a source of electricity, compressed gas and liquid. The tip of the hand piece, which provides the jet of gas with solid powder particles, is comprised of a converging-diverging nozzle, where the converging-diverging nozzle inlet is operably coupled with a source of compressed gas and the diverging portion of this nozzle is operably coupled with a powder feeder. Compressed gas passing through this converging-diverging nozzle accelerates in the diverging portion to supersonic speed and creates a vacuum environment. This results in aspiration of solid powder particles from the feeder into this supersonic jet of gas. where the particles become entrained and accelerate to the nozzle outlet.

### TECHNICAL FIELD

The present invention relates generally to the field of devices for propelling (blasting) solid powder particles which intend to polish or abrade a target surface. More particularly, the present invention pertains to an improved dental abrasion apparatus and method for generating a jet of air mixed with solid powder particles. The intent is to employ this jet in a dental practice for the treatment of teeth, and teeth surfaces including cleaning, polishing, abrading, etching, removing surface discoloration or bacterial plaque, and teeth preparation before bonding.

### BACKGROUND ART

In dentistry, apparatus for propelling solid particles with jet of air, intended to polish or abrade the surface of a tooth, have been known for decades. With patented inventions such as Maurer U.S. Pat. No. 1,664,369, and Black U.S. Pat. No. 2,696,049, as well as many others, these kinds of surface treatments are well known in dentistry.

Some invented apparatus provide air abrasion systems with both delivery and evacuation of abrasive particles, such as Brassil U.S. Pat. No. 6,106,288. Some serve as an attachment to standard dental equipment, such as Groman U.S. Pat. No. 8,668,552, Nesbitt U.S. Pat. No. 7,993,135, and Fernwood U.S. Pat. No. 4,941,298. Some devices incorporate water to limit the spread of sprayed abrasive particles, such as Harris U.S. Pat. No. 6,729,942, Grant U.S. Pat. No. 6,676,409, Gallant U.S. Pat. No. 4,522,597, and Black U.S. Pat. No. 3,972,123. Others implement converging-diverging nozzles to increase the speed of the mixture of propellant air and abrasive particles, such as LaSalle U.S. Pat No. 6,273,789.

Many recently developed apparatus are associated with their own particular operational console of such size, that it is difficult to accommodate these devices and associated consoles in the already crowded space allotted to equipment mandatory for the practice of dentistry. An additional significant problem with currently developed apparatus is the clogging of abrasive material. It has also been found that different abrasive materials are more suited for particular dental procedures. Unfortunately, conventional air abrasion apparatus do not effectively switch between different materials of differing particle sizes and characteristics. The present invention has been devised to obviate the disadvantages of prior apparatus employed to accomplish the aforementioned corrective procedures, and to provide an improved apparatus of relatively simple principle and design, that can be easily integrated within an existing dental clinic system.

### DISCLOSURE OF THE INVENTION

The primary objective of this invention is to provide a dental apparatus that can be integrated within an existing dental clinic system or which can be a part of a new dental system. The major component of this dental apparatus is a hand piece similar in shape, size, and weight to existing high-speed dental hand pieces. It is coupled to a supply cable, permanently attached to the dental instrument supply unit, and is operably coupled to a powder feeder. The hand piece is held in one cavity of a traditional dental instrument holder or on an instrument pad of a continental dental supply control, as seen with other standard dental instruments. By removing it from the holder cavity or from the instrument pad, the operator activates this dental apparatus to standby mode. By pressing a foot pedal, the operator activates the process of abrasion or polishing. The operator can control the intensity of the abrasion or polishing process by applying varying pressure to the foot pedal, in a similar way to the control of rotation speed of the dental hand piece drill. Optionally, when the hand piece is equipped with an adapter, this dental apparatus can be connected to a standard dental instrument supply fitting that provides the air, water and electricity to dental instruments and becomes a removable attachment of the dental system. Together, this dental apparatus, along with the adapted dental clinic equipment, create an integrated and cost-effective air abrasion and air polishing dental system. This dental apparatus is easy to operate and maintain with a wide flexibility for changing process parameters and materials used. Due to its compactness, it occupies a minimal amount of space.

Another objective of this invention is to provide a dental apparatus which will effectively use low pressure air available in a dental clinic (around 60 Psi) without sacrificing the efficiency of the air abrasion and polishing processes. This dental apparatus hand piece has a tip equipped with a converging-diverging nozzle where the air jet velocity increases in the direction of flow to supersonic speed. To reduce the creation of dust clouds during the abrasion or polishing processes, this dental apparatus is connected to a water supply, and is equipped with a water nozzle, where a water hollow cone curtain is created. This water hollow cone curtain covers the jet of air mixed with solid powder particles when they exit the nozzle outlet and dampens the creation of dust clouds.

Safety of air abrasion and air polishing in dental procedures is a crucial issue. As such, an additional important objective of this invention is to provide a dental apparatus with a manually operable, normally open safety valve. When this dental apparatus is activated, the safety valve must be closed in order to generate the desired jet of air mixed with solid powder particles. The abrasion or polishing procedures can be aborted by deactivating the foot pedal. The safety valve is an additional item located on the handle and works via finger touch to rapidly stop or activate feeding solid powder particles into the jet of air. When this safety valve is left open, a jet of gas covered by the water hollow cone curtain is generated and supplied to the treated surface, without the addition of solid powder particles. Additionally, when the water supply switch is turned off, only the jet of air is supplied. This normally open safety valve improves safety by eliminating accidental activation of this apparatus, and creates the possibility for cleaning and drying of the treated area before examination and without the necessity of using other dental instruments. An additional objective of this invention is to provide a dental apparatus with a manually operable process intensification valve. The process intensification valve is located near the safety valve and works via finger touch action. It can be used to change the intensity of the abrasion or polishing processes by changing the volume ratio of solid powder particles to the air in the jet of the mixture. When this valve is used, the vacuum volume in the feeder drops and less powder particles enter into the jet of air.

Another objective of this invention is to provide a dental apparatus that is connected to the powder feeder operably coupled with a diverging side of the converging-diverging nozzle placed within the tip of the hand piece. A properly located powder injection point at the diverging side of the converging-diverging nozzle permits the maintenance of a vacuum environment in the powder feeder and enables the use of atmospheric air to feed solid powder particles from the feeder into the diverging side of the converging-diverging nozzle. The powder feeder is mounted in the feeder holder, which is attached to the dental instrument supply unit. One or more powder feeders can be accommodated in the feeder holder, where one of the powder feeders can be operably coupled with the diverging side of the converging-diverging nozzle. The majority of solid powder particles used in dentistry are very sensitive to moisture, which they absorb from the surrounding air leading to potential feeding problems. For this reason, when powder feeder is not in use, the solid powder particles in it have to be isolated from the surrounding air. The powder feeder allows efficient feeding of a wide variety of different materials of differing particle sizes and characteristics that can be easily and quickly refilled or exchanged. Thus, this dental apparatus can be used for air abrasion when connected to the powder feeder with hard and abrasive particles, such as aluminum oxide. For an air polishing procedure, mild particles such as sodium bicarbonate, aluminum trihydroxide, calcium sodium phosphosilicate, calcium carbonate, erythrital or glycine can be used within the powder feeder.

An additional objective of this invention is to provide a dental apparatus with a hand piece equipped with fiber optics which transmits light from an LED lamp to the nozzle outlet.

Another objective of this invention is to provide a dental apparatus comprising a tip attached to the front end of the handle with a variety of different orifice sizes of converging-diverging nozzles. The tip is secured to the handle by a nut and marked by a colored rubber washer to identify its size. This feature helps in choosing the appropriate setup of this dental apparatus for differing dental procedures.

The objectives and related advantages of this invention become more apparent in the following detailed description and accompanying drawings.

### BRIEF DESCRIPTION OF THE DRAWINGS

FIG. 1 is a schematic representation of the present invention,

FIG. 2 is a schematic representation of the optional embodiment of the present invention,

FIG. 3 is a cross section of the feeder of the present invention,

FIG. 4 is a view of the powder feeder assembly of the present invention, and

FIG. 5 is a cross section view of the powder feeder assembly of the present invention.

### BEST MODE FOR CARRYING OUT THE INVENTION

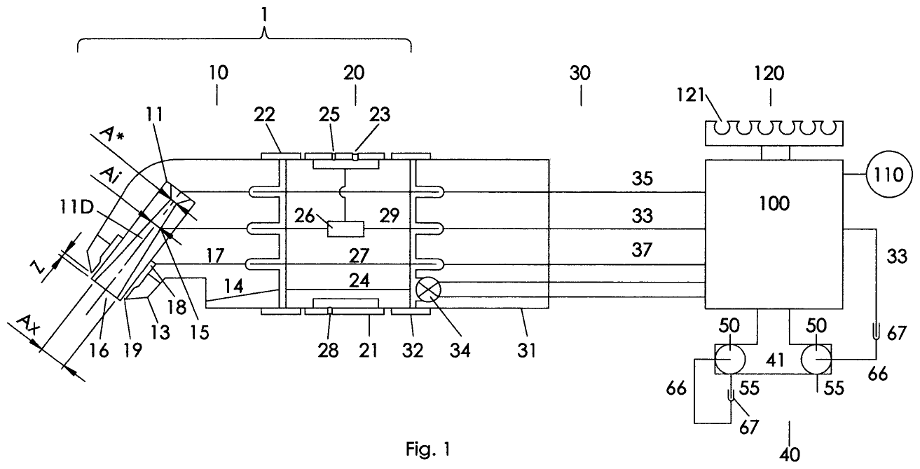

An embodiment of the dental apparatus according to the invention is shown in FIG. 1, which is a schematic representation of the invention where a hand piece 1 and a powder feeder assembly 40 are integrated within a dental clinic system by a powder feeder holder 41 and a supply conduit 30 permanently attached to a dental instrument supply unit 100, for a source of pressurized propellant gas, pressurized liquid and electricity. While helium, nitrogen, carbon dioxide, and other gases may be used as the propellant and water with antibacterial/aroma/flavor additives as the liquid, in accordance with the present invention, for the sake of simplicity the dental apparatus is described below using air as the propellant and water as the liquid.

The major component of the hand piece 1 is a handle 20 with a removable tip 10 attachment at one end, and at the other end the supply conduit 30. Connections between them are sealed with rubber pads (not shown) and secured by nuts 22 and 32. The hand piece 1 is similar in shape, size, and weight to existing dental high-speed hand pieces, and is held directly in an existing instrument holder cavity 121 of a traditional dental instrument holder 120 or on an instrument pad (not shown) of a continental dental supply control. The handle 20 and the tip 10 can be autoclaved. By removing the hand piece 1 from the instrument holder cavity 121 or from the instrument pad (not shown), the operator activates this dental apparatus to standby mode. By pressing a foot pedal 110, the operator activates the flow of compressed air from the dental instrument's supply unit 100 through a flexible air tube 35 of the supply conduit 30, to the handle 20, then through a converging-diverging nozzle 11, that is found within the tip 10 and out through a nozzle outlet 16. The compressed air flowing through the converging-diverging nozzle 11 accelerates in the diverging side 11 D to supersonic speeds and creates a vacuum environment within it. This results in the aspiration of the solid powder particles from a powder feeder 50 through powder conduits 66 and 33 to a powder tube 29, then to a powder channel insert 26 and into a supersonic jet of air at an injection point 15, where they become entrained and accelerate to the nozzle outlet 16. The supersonic jet of air accelerates injected solid powder particles, imparts them with kinetic energy, which allows for polishing or abrading when they strike the target tooth surface. The amount of vacuum created by the converging-diverging nozzle 11 depends on the air inlet pressure and the converging-diverging nozzle 11 configuration of an orifice size, the diverging side 11D exit size, and the position of the injection point 15 along the diverging side 11D. Numerous tests and experiments have shown that in a miniature nozzle system, where a low compressed air inlet pressure is used in order for the powder to be effectively transported by atmospheric pressure and injected into the supersonic jet of air, the position of the injection point 15 and size of the diverging side 11D exit can be calculated by the following relations:

$$Ai / A * \geq 3 P and Ax / A * \geq 8.5 P$$

Where:
Ai—is the cross-sectional area of the diverging side of the nozzle at the injection point,
A*—is the cross-sectional area of the converging-diverging nozzle orifice,
Ax—is the cross-sectional area of the nozzle exit,
P—is the nozzle inlet pressure (MPa).

The abrasion or polishing process can be aborted by the deactivation of the foot pedal 110, but to avoid an accidental ejection of the solid powder particles, the handle 20 is equipped with a swivel ring 21 which has a first opening 23 across its wall which is in fluid communication with the powder channel insert 26. This first opening 23, together with the operator's index finger, create a safety valve. The swivel ring 21 can be rotated over the handle 20, thus, the position of the first opening 23 can be adjusted, when needed, to hold the hand piece 1 in the most ergonomic position when the dental apparatus is in use. When the dental apparatus is activated to proceed with the abrasion or polishing process, the first opening 23 must be covered. If not, the vacuum environment in the powder tube 29, powder conduits 33 and 66, and the interior of the powder feeder 50 is broken and powder particles are not fed into the supersonic jet of air. The swivel ring 21 has a second opening 25 located near the first opening 23 which is also in fluid communication with the powder channel insert 26 and which, with the operator's index finger, creates a process intensification valve. When the second opening 25 is not covered, it results in the lowering of the vacuum intensity in the powder feeder 50 interior and changing the volume ratio of solid powder particles to propellant air in the jet of air and solid powder particles. The swivel ring 21 has a third opening 28 which is also in fluid communication with the powder channel insert 26 and which stabilizes the flow of powder particles from the powder feeder 50 to the injection point 15. The speed of the supersonic jet of air, the size of the abrasive particles, as well as the volume ratio of abrasive particles to air, play a critical role in the achievement of the desired result of the abrasion or polishing process. For tooth surface finishing and cleaning, a higher proportion of abrasives in the supersonic jet of air achieve the desired process objective more efficiently, while for drilling and cutting, more air and less powder is recommended. To make the process of polishing or abrasion more economical, the tips 10 are equipped with a plurality of different orifice sizes of the converging-diverging nozzle 11 within them. The operator has the option to choose a different tip 10 size for different dental procedures. The tip 10 is secured to the handle 20 by the nut 22 and marked by a colored rubber washer (not shown) to identify the orifice size, where tips with larger converging-diverging nozzle 11 orifice sizes are for less precise but faster cleaning and tips with smaller nozzle orifice sizes are recommended for more precise and accurate functions.

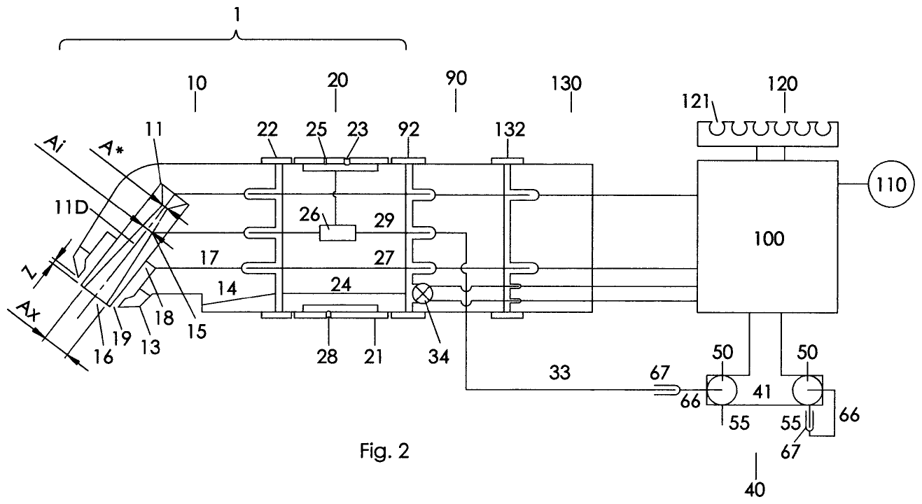

To reduce the creation of dust clouds during the abrasion or polishing processes, the tip 10 is equipped with a water nozzle 13 which is in fluid communication with the dental instrument supply unit 100 source of compressed water by a flexible water tube 37 of the supply conduit 30 and a water tube 27 incorporated in the handle 20 and a water channel 17 inside the tip 10. The water nozzle 13 is installed into the exit of the tip 10 and is disposed coaxially over the outside diameter of the converging-diverging nozzle 11. Together, they create a water annular discharge orifice 19 which is slightly upstream of the nozzle outlet 16, distanced Z (FIG. 1 and FIG. 2) most advantageously from 0.5 mm to 1.5 mm. Compressed water flowing through the water channel 17 enters a water chamber 18 tangentially and creates a stream of water which flows in a swirling manner. This swirling motion plays a very important role in the creation of the hollow cone water curtain which covers the jet of air with the solid powder particle mixture when it is discharged from the converging-diverging nozzle 11 at the nozzle outlet 16. The shape and intensity of this water curtain can be adjusted by changing the volume of compressed water supply using the water regulator located in the dental instruments supply unit 100.

To increase the visibility of the treated surface, this dental apparatus is equipped with an LED lamp 34 installed inside a connector 31 of the supply conduit 30 that is attached to the dental instrument supply unit 100 source of electricity. It generates light transmitted through a fiber optic rod 24 inside the handle 20 and a fiber optic rod 14 in the tip 10 to the nozzle outlet 16.

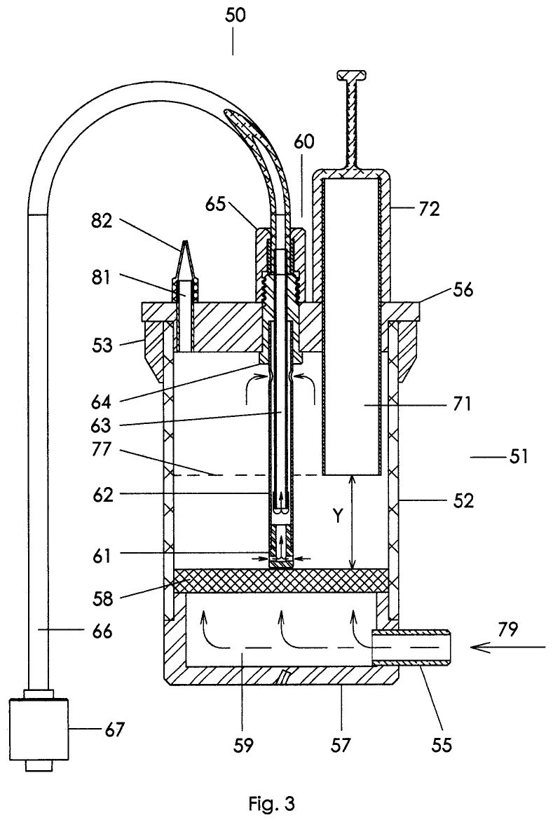

To allow the efficient feeding of a wide variety of different powder materials of differing particle sizes and characteristics, a floating bed powder feeder is used. Referring to FIG. 3, the powder feeder 50 is comprised of a housing 51 formed by a top cover 56, a bottom cover 57, a cylindrical side wall 52, and a fluidizing plate 58 mounted to the cylindrical side wall 52 within the interior of the housing 51. The fluidizing plate 58 is placed above the bottom cover 57 and forms a fluidizing air chamber 59 which is connected via a fluidizing air inlet tube 55 with surrounding air 79. The top cover 56 is comprised of a powder pick-up tube system 60, an inlet tube 71 and a vent tube 81—they communicate with the interior of the housing 51. The inlet tube 71 is used for refilling powder and is covered by a cap 72. To identify powder particles in a powder feeder 50, feeders are equipped with colour coded rings 53 where their colour matches the colour of the caps 72. The distance Y between the fluidizing plate 58 and the lower portion of the inlet tube 71 determines the maximal level of powder 77 that can be filled into the powder feeder 50. The cylindrical side wall 52 is transparent which makes the interior of the housing 51 visible. The powder pick-up tube system 60 is comprised of a tip 61 mounted into an outer pick-up tube 62 which is inserted into a tube holder 64 and secured by a nut 65 to the top cover 56, then to an inner pick-up tube 63, which is connected with the powder conduit 66. When the abrasion or polishing process is activated and the interior of the housing 51 vacuum environment is established, the air from the fluidizing air chamber 59 is directed upwardly through the fluidizing plate 58 to form a fluidized bed of powder suspended above the fluidizing plate 58. The atmospheric air transmits particulate powder material mixed with air from the fluidized bed through the powder pick-up tube system 60 and the powder conduit 66, continuing through the dental apparatus (shown in FIGS. 1 & 2) to the injection point 15. The vent tube 81 is equipped with a non-return duckbill check valve 82 which covers the vent tube 81 and vents the interior of the housing 51 in order to avoid blocking pores of the fluidizing plate 58 when the nozzle outlet 16 (FIGS. 1 & 2) is blocked and positive pressure is created in the interior of the housing 51.

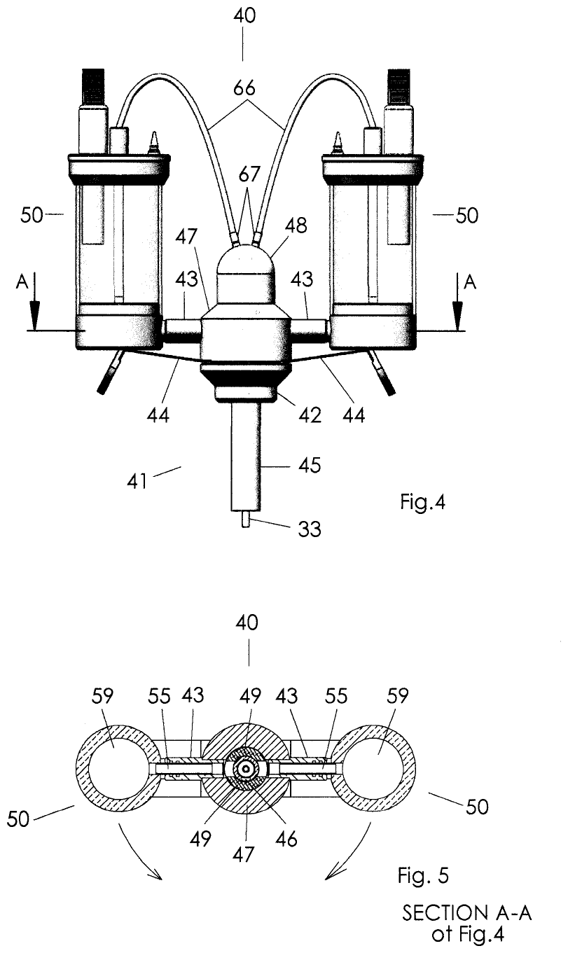

Referring to FIG. 4 and FIG. 5 the powder feeder assembly 40 of the invention is shown, where the powder feeder holder 41 accommodates two powder feeders 50. A base 42 of the powder feeder holder 41 is installed by an extension tube 45 and a bracket (not shown) onto the dental instrument supply unit 100 and holds a swivel ring 47 with hollow holder arms 43 installed into it. The swivel ring 47, which can be rotated over the base 42 is secured in place by a top nut 48 and a rotation stop pin (not shown) where the connection between the base 42 and the swivel ring 47 is sealed by rubber pads 49. Each of the powder feeders 50 is installed onto the powder feeder holder 41 by inserting the fluidizing air inlet tube 55 of the feeder 50 into the hollow holder arm 43 and connecting powder conduits 66 and 33 by a quick disconnect fitting 67, where a quick connection between the fluidizing air inlet tube 55 and the hollow holder arm 43 is sealed by O-rings (not shown) and secured by a spring latch 44. The powder feeder assembly 40, as shown in FIG. 4 and FIG. 5 is in a neutral position where the connection between fluidized air chambers 59 of the powder feeder 50 and the surrounding air are blocked. One at a time, the powder feeder 50 can be activated by rotating the swivel ring 47 left or right to the rotation stop pin (not shown), which unblocks the connection between the fluidized air chamber 59 with the surrounding air by matching a hole through one of the hollow holder arms 43 with an opening 46 across the base 42 side wall. The activated powder feeder 50 is exposed to the front of the dental instrument supply unit.

An optional embodiment of the dental apparatus according to the invention is shown in FIG. 2 which is a schematic representation of the invention, where an adaptor 90 is connected to the hand piece 1, to the powder feeder 50 of the powder feeder assembly 40, and to a dental instrument supply fitting 130 which furnishes air, water and electricity to it. The adaptor 90 is equipped with the LED lamp 34 installed inside of it, which generates light transmitted through the fiber optic rod 24 inside the handle 20 and the fiber optic rod 14 in the tip 10, and to the nozzle outlet 16. The connections between the adaptor 90, the hand piece 1, and the dental instrument supply fitting 130 are sealed with rubber pads (not shown) and secured by nuts 92 and 132. This dental apparatus, with the hand piece 1 and the powder feeder assembly 40 (which are sufficiently described above) together with the adaptor 90, and the dental instrument supply fitting 130, become a removable attachment of the dental system.

### INDUSTRIAL APPLICABILITY

As has been demonstrated, the present invention provides a new and improved air abrasion and air polishing apparatus which can be integrated within the standard dental system and together create a very complex but effective air abrasion and air polishing dental system which is easy to operate and maintain with a wide flexibility for changing process parameters, materials used, and more effective switching between abrasive materials.

It is intended that the invention not be limited to the particular embodiment disclosed as the best mode contemplated for carrying out this invention, but that the invention will include all embodiments falling within the scope of the appended claims.

1. A dental apparatus for generating a jet of gas mixed with solid powder particles, the dental apparatus comprising:
a compressed gas source;
a converging-diverging nozzle within a tip for directing the jet of gas mixed with solid powder particles against a surface, where an inlet of the converging-diverging nozzle is operably coupled to the compressed gas source;
a handle which holds at one end the tip and a supply cable connected to the compressed gas source at the other end, where the connections between them are sealed and secured, and fluid communication within them is established; and a powder feeder operably coupled with a diverging side of the converging-diverging nozzle for feeding solid powder particles into the jet of gas.

2. The dental apparatus of claim 1, further comprising:
a swivel ring which has a rotational connection with the handle and has a first opening across its wall which is in fluid communication with a powder channel along the handle's elongated interior; and
a manually operated safety valve which works via operator finger covering/unveiling of the swivel ring's first opening for feeding/not feeding solid powder particles into the jet of gas.

3. The dental apparatus of claim 2 where the swivel ring has a second opening across its wall which together with finger covering/unveiling create a manually operated process intensification valve which can be used to change the volume ratio of solid powder particles to gas in the jet of the mixture.

4. The dental apparatus of claim 3 where the swivel ring has a third opening across its wall for stabilizing the flow of powder particles in the powder channel.

5. The dental apparatus of claim 1, further comprising:
a compressed liquid source;
a liquid whirl chamber created inside the tip where a liquid inlet passage connected with the compressed liquid source is introduced tangentially which force a stream of the compressed liquid to flow in a swirling manner; and
a liquid nozzle incorporated into the tip and disposed coaxially with the outside wall of the converging-diverging nozzle for the creation of a liquid annular discharge orifice which is slightly upstream of the converging diverging nozzle outlet.

6. The dental apparatus of claim 1, further comprising:
a source of electricity; and
an LED lamp installed inside a fitting connector of the supply cable connected to the source of electricity and optically connected to fiber optics installed inside the handle and the tip which transmits light to the converging-diverging nozzle outlet.

7. The dental apparatus of claim 1, where the compressed gas source, the compressed liquid source, and the source of electricity is a dental supply unit.

8. A dental apparatus for generating a jet of gas mixed with solid powder particles, the dental apparatus comprising:
an adaptor attached to a dental instrument supply fitting that is source of compressed gas, compressed liquid and electricity;
a converging-diverging nozzle within a tip for directing the jet of gas mixed with solid powder particles against a surface, where the inlet of the converging-diverging nozzle is operably coupled to the compressed gas;
a handle which holds at one end the tip and at the other end the adaptor, where connections between them are sealed and secured, and fluid communication within them is established; and
a powder feeder operably coupled with a diverging side of the converging-diverging nozzle for feeding solid powder particles into the jet of gas.

9. The dental apparatus of claim 8, further comprising:
a swivel ring which has a rotational connection with the handle and has a first opening across its wall which is in fluid communication with a powder channel along the handle's elongated interior; and
a manually operated safety valve which works via operator finger covering/unveiling of the swivel ring's first opening for feeding/not feeding solid powder particles into the jet of gas.

10. The dental apparatus of claim 9 where the swivel ring has a second opening across its wall which together with finger covering/unveiling create a manually operated process intensification valve which can be used to change the volume ratio of solid powder particles to gas in the jet of the mixture.

11. The dental apparatus of claim 10 where the swivel ring has a third opening across its wall for stabilizing the flow of powder particles in the powder channel.

12. The dental apparatus of claim 8, further comprising:
a liquid whirl chamber created inside the tip where a liquid inlet passage connected with the compressed liquid source is introduced tangentially which forces a stream of the compressed liquid to flow in a swirling manner; and
a liquid nozzle incorporated into the tip and disposed coaxially with the outside wall of the converging-diverging nozzle for the creation of a liquid annular discharge orifice which is slightly upstream of the converging diverging nozzle outlet.

13. The dental apparatus of claim 8, further comprising:
an LED lamp installed inside the adaptor connected to the source of electricity and optically connected to fiber optics installed inside the handle and the tip which transmits light to the converging-diverging nozzle outlet.

14. The dental apparatus of claim 1, where the powder feeder is comprised of:
a vacuum source;
a housing with a powder pick-up tube system which communicates with the interior of the housing and is coupled to the vacuum source and transmits powder particles mixed with air out of the housing;
a fluidizing plate installed inside the housing, wherein the bottom of the fluidizing plate is exposed to the atmospheric air which fluidizes powder particles suspended above the fluidizing plate when a vacuum environment is established inside the housing;
a fluidizing air inlet tube incorporated into the bottom of the housing which is used for holding the feeder in a powder feeder holder and connects the bottom of the fluidizing plate with the surrounding air;
an inlet tube incorporated into the top of the housing unit which is in fluid communication with the interior of the housing, is covered by a cap, is used to refill powder particles and prevents overflow of them; and
a vent tube incorporated into the top of the housing which is in fluid communication with the interior of the housing and is covered by a non-return check valve which vents the interior of the housing when positive pressure is created in it.

15. The dental apparatus of claim 14, where the powder feeder holder is comprised of:
a base with an opening in its side wall;
a swivel body with hollow holding arms installed into it for accommodation of one or more feeders, where the rotational connection with the base is secured and sealed and one at a time, a hole through the hollow holding arm is matched with the opening in the base side wall when the swivel body is rotated left or right to a stop, which activates fluid communication between the bottom of the fluidizing plate with the surrounding air; and
a spring latch which holds and secures the powder feeder in place.

16. The dental apparatus of claim 5 where the compressed gas source, the compressed liquid source, and the source of electricity is a dental supply unit.

17. The dental apparatus of claim 6 where the compressed gas source, the compressed liquid source, and the source of electricity is a dental supply unit.

18. The dental apparatus of claim 8, where the powder feeder is comprised of:
a vacuum source;
a housing with a powder pick-up tube system which communicates with the interior of the housing and is coupled to the vacuum source and transmits powder particles mixed with air out of the housing;
a fluidizing plate installed inside the housing, wherein the bottom of the fluidizing plate is exposed to the atmospheric air which fluidizes powder particles suspended above the fluidizing plate when a vacuum environment is established inside the housing;
a fluidizing air inlet tube incorporated into the bottom of the housing which is used for holding the feeder in a powder feeder holder and connects the bottom of the fluidizing plate with the surrounding air;
an inlet tube incorporated into the top of the housing unit which is in fluid communication with the interior of the housing, is covered by a cap, is used to refill powder particles and prevents overflow of them; and
a vent tube incorporated into the top of the housing which is in fluid communication with the interior of the housing and is covered by a non-return check valve which vents the interior of the housing when positive pressure is created in it.
